(19) United States
(12) Patent Application Publication
(10) Pub. No.: US 2025/0075052 A1
(43) Pub. Date: Mar. 6, 2025

(54) NETWORK POLYMER AND MANUFACTURING METHOD THEREOF

(71) Applicant: KOREA INSTITUTE OF SCIENCE AND TECHNOLOGY, Seoul (KR)

(72) Inventors: Hae Jung SON, Seoul (KR); Hyunjung JIN, Seoul (KR); Kyuyeon KIM, Seoul (KR); Jinhyeong RHEE, Seoul (KR); Kyeongmin KIM, Seoul (KR); Eul-Yong SHIN, Seoul (KR); Hyeonggeun YU, Seoul (KR); Jai Kyeong KIM, Seoul (KR); Jin Young KIM, Seoul (KR)

(21) Appl. No.: 18/398,677

(22) Filed: Dec. 28, 2023

(30) Foreign Application Priority Data
Sep. 5, 2023 (KR) 10-2023-0117737

(51) Int. Cl. C08J 11/20; C08J 11/14; H01L 31/072; H10K 30/30

(52) U.S. Cl. CPC C08J 11/20; C08J 11/14; H10F 10/16; H10K 30/30

(57) ABSTRACT

The present disclosure relates to a network polymer and a manufacturing method thereof. An object of one aspect of the present invention is to provide a network polymer that can recover and recycle a key monomer from a polymer with excellent decomposability and is formed therefrom to have excellent mechanical and electrical properties, and a method of manufacturing the same. The network polymer according to one embodiment of the present invention is recycled from a polymer with excellent decomposability and thus is eco-friendly, and exhibits the effect of having excellent mechanical and electrical properties.

### CROSS REFERENCE TO RELATED APPLICATION

The present application claims priority to Korean Patent Application No. 10-2023-0117737, filed Sep. 5, 2023, the entire contents of which is incorporated herein for all purposes by this reference.

### BACKGROUND OF THE INVENTION

### Field of the Invention

Disclosed herein are a network polymer and a method of manufacturing the same.

### Description of the Related Art

This invention was made with the support of the Ministry of Science and ICT under Project No. 1711173310, which was conducted under the research project entitled “Renewable energy technologies and disaster monitoring for countering climate change” within the project named “Nano Material Technology Development” under the management of the National Research Foundation of Korea, from Jan. 1, 2022 to Dec. 31, 2022.

This invention was made with the support of the Ministry of Science and ICT under Project No. 1711155944, which was conducted under the research project entitled “Development of technology for stretchable organic photovoltaic modules with high stability” within the project named “Nano Material Technology Development” under the management of the National Research Foundation of Korea, from Jan. 1, 2022 to Dec. 31, 2022.

This invention was made with the support of the Ministry of Science and ICT under Project No. 1711157617, which was conducted under the research project entitled “Development of solution processed-based technology for stability of rollable optoelectronic devices” within the project named “Nano Material Technology Development” under the management of the National Research Foundation of Korea, from Jan. 1, 2022 to Dec. 31, 2022.

This invention was made with the support of the Ministry of Science and ICT under Project No. 1055001146, which was conducted under the research project entitled “Research of key element technologies for highly efficient, 100 cm² photoelectrochemical cell-based water splitting” within the project named “Nano Material Technology Development” under the management of the National Research Foundation of Korea, from Jan. 1, 2023 to Dec. 31, 2023.

Solar cells are photoelectric conversion devices that convert solar energy into electrical energy, and are in the spotlight as a next-generation energy resource. The solar cells can be broadly divided into inorganic solar cells and organic solar cells, wherein depending on the structure of the photoactive layer, the organic solar cells are divided into a bi-layer p-n junction type in which a p-type semiconductor and an n-type semiconductor are formed as separate layers, and a bulk heterojunction (BHJ) type in which a p-type semiconductor and an n-type semiconductor are mixed. In 1986, C. Tang of Eastman Kodak first suggested the possibility of practicalizing a solar cell with a heterojunction structure using copper phthalocyanine (CuPc) and perylene tetra-carboxylic derivative. Afterward, in early 1990, the Heeger group reported a solar cell that generates electricity using a mixed film of conjugated polymer and fullerene derivative as a photoactive layer, and improved the efficiency to 7-8% by developing a fullerene derivative (PCBM) modified from fullerene. Since then, various studies have been continuously conducted to obtain highly efficient organic solar cells, and photoelectric conversion efficiency is currently increasing significantly.

Nevertheless, the organic solar cells have the problem of being easily destroyed or having deteriorated electrical properties due to defects such as cracks in the photoactive layer caused by various external stimuli (or forces) such as ultraviolet rays, moisture, air, or strain. In addition, there is a problem of environmental pollution such as the generation of toxic chemicals due to improper disposal of electronic and electrical waste, so it is necessary to develop organic semiconductors with decomposability and develop devices with excellent properties therefrom.

### SUMMARY OF THE INVENTION

### Technical Problem

An object of one aspect of the present invention is to provide a network polymer that can recover and recycle a key monomer from a polymer with excellent decomposability and is formed therefrom to have excellent mechanical and electrical properties, and a method of manufacturing the same.

### Technical Solution

In one aspect of the present invention, the invention provides a network polymer formed from a monomer recovered from a recyclable conjugated polymer.

In another aspect, the present invention provides an organic electronic device comprising the network polymer.

In another aspect, the present invention provides a method for manufacturing the network polymer, the method comprising the steps of: hydrolyzing a recyclable conjugated polymer to recover a monomer; and forming a network polymer by covalent bonding of the monomer and a cross-linking agent.

### Advantageous Effects

The network polymer according to one embodiment of the present invention is recycled from a polymer with excellent decomposability and thus is eco-friendly, and exhibits the effect of having excellent mechanical and electrical properties.

### BRIEF DESCRIPTION OF THE DRAWINGS

FIG. 1 is an image showing a process of recovering a monomer from a conjugated polymer according to an embodiment of the present invention.

FIG. 2 is an image showing a process of manufacturing a network polymer according to an embodiment of the present invention.

FIG. 3 is an image showing the results of evaluating the formation capacity of a network polymer according to an embodiment of the present invention.

FIGS. 4 and 5 are UV-vis spectrum graphs showing the results of evaluating the recovery capacity of a monomer according to an embodiment of the present invention.

FIG. 6 is an NMR graph showing the results of evaluating the recovery capacity of a monomer according to an embodiment of the present invention.

FIG. 7 is a UV-vis spectrum graph showing the results of evaluating the recovery capacity of a monomer according to an embodiment of the present invention.

FIG. 8 is a graph showing a current density-voltage (J-V) curve of an organic solar cell according to an embodiment of the present invention.

FIG. 9 is a graph showing the results of evaluating the thermal stability of an organic solar cell according to an embodiment of the present invention.

FIG. 10 is a graph showing the results of evaluating the mechanical properties of a network polymer according to an embodiment of the present invention.

### DETAILED DESCRIPTION OF THE INVENTION

Hereinafter, preferred embodiments of the present invention will be described in detail with reference to the attached drawings.

The embodiments of the present invention disclosed herein are illustrated for the purpose of explanation only, and the embodiments of the present invention may be implemented in various forms and should not be construed as being limited to the embodiments described herein. The present invention can be subject to various changes and take various forms, whereby the embodiments are not intended to limit the present invention to the specific disclosed forms, and should be understood to include all modifications, equivalents, and substitutes included in the spirit and technical scope of the present invention.

In this specification, when a part is described as “including” a certain component, it means that other components may be further included, rather than excluded, unless otherwise stated.

Throughout the specification, similar parts are given the same reference numerals. Throughout the specification, when a part such as a layer, film, region, plate, etc. is said to be “on” or “above” another part, this includes not only the case where the part is directly above the another part, but also the case where there is still another part between them. Throughout the specification, terms such as first, second, etc. may be used to describe various components, but the components should not be limited by the terms. The terms are used only for the purpose of distinguishing one component from another.

### Network Polymer

In one aspect of the present invention, the invention provides a network polymer formed from a monomer recovered from a recyclable conjugated polymer.

There is a problem of environmental pollution such as the generation of toxic chemicals due to improper disposal of electronic and electrical waste from organic electronic devices, so it is necessary to develop organic semiconductors with decomposability and develop devices with excellent properties therefrom. Accordingly, the present inventors have developed a recyclable conjugated polymer, and also developed a network polymer that is recycled from the conjugated polymer and thus is eco-friendly, and has excellent mechanical and electrical properties.

In one embodiment, the conjugated polymer has a number average molecular weight of 13.4 kDa. The conjugated polymer has a polydisperisty (PDI) (Mw/Mn) of 1.55.

In one embodiment, the recyclable conjugated polymer includes an imine bond, and the monomer is a dialdehyde compound.

In one embodiment, the recyclable conjugated polymer includes a repeating unit represented by Formula 1 below.

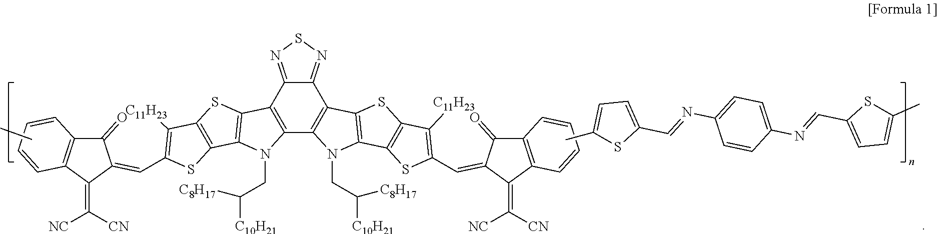

In Formula 1, n is an integer ranging from 1 to 1000.

In one embodiment, the monomer is a compound represented by Formula 2 below.

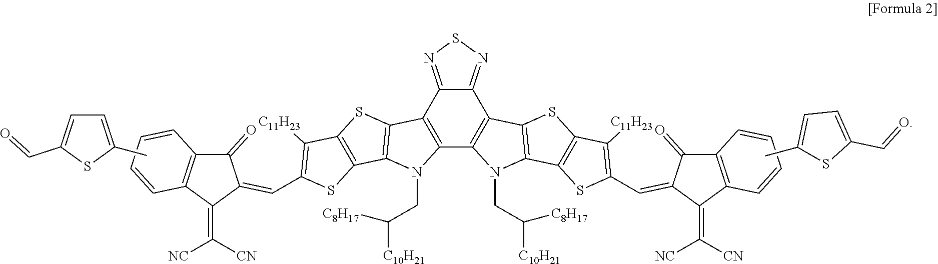

In one embodiment, the network polymer includes a repeating unit represented by Formula 3 below.

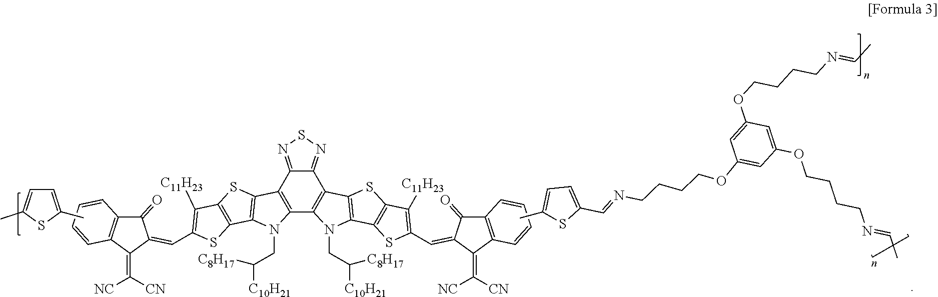

In Formula 3, n is an integer ranging from 1 to 1000.

### Organic Electronic Device

In another aspect, the present invention provides an organic electronic device comprising the network polymer.

In one embodiment, the organic electronic device is any one selected from an organic solar cell, an organic thin film transistor, and an organic light emitting diode.

The organic solar cell is not particularly limited as long as it has the structure of a general organic solar cell, and basically includes a first electrode, a photoactive layer, and a second electrode, but may further include components such as a substrate and a buffer layer depending on the use or need.

For the first electrode, indium-tin oxide (ITO), fluorine doped tin oxide (FTO), ZnO—(Ga₂O₃ or Al₂O₃), SnO₂—Sb₂O₃, etc. may be used, and more preferably ITO is used.

A buffer layer may be further included on top of the first electrode, wherein the buffer layer may use zinc oxide (ZnO) or polyethyleneimine ethoxylated (PEIE) to improve electron injection. In this case, the buffer layer may be formed through a method such as spin coating.

A photoactive layer is stacked on top of the buffer layer. The network polymer is included in the photoactive layer.

The material used for the second electrode may be any one selected from the group consisting of gold, aluminum, copper, silver, molybdenum oxide (MoO₃), or a combination thereof, and is preferably silver or silver/molybdenum oxide (MoO₃).

In one embodiment, the organic electronic device is a bulk heterojunction (BHJ) type solar cell, and the network polymer is included in a photoactive layer of the bulk heterojunction type solar cell.

The bulk heterojunction (BHJ) type solar cell, a kind of organic solar cell, is a type of solar cell in which the active layer generating electrons and holes is manufactured by mixing an electron donor and an electron acceptor in order to generate as many electron/hole pairs as possible when irradiated with sunlight.

The organic electronic device according to the present invention is recycled from the conjugated polymer and thus is eco-friendly, and has excellent mechanical and electrical properties.

### Network Polymer Manufacturing Method

In another aspect, the present invention provides a method for manufacturing the network polymer, the method comprising the steps of: hydrolyzing a recyclable conjugated polymer to recover a monomer; and forming a network polymer by covalent bonding of the monomer and a cross-linking agent.

In the step of recovering the monomer, the recovered monomer does not contain impurities and thus can be recycled without additional purification.

According to an embodiment, in the step of forming the network polymer, the network polymer is prepared on a thin film from the monomer without a catalyst.

In one embodiment, the network polymer is used as a blend with an electron donor compound. As the electron donor compound, a polymer compound such as poly [[4,8-bis[5-(2-ethylhexyl)-2-thienyl]benzo[1,2-b: 4,5-b′]dithiophene-2,6-diyl]-2,5-thiophenediyl[5,7-bis(2-ethylhexyl)-4,8-dioxo-4H,8H-benzo[1,2-c: 4,5-c′]dithiophene-1,3-diyl]]polymer (abbreviated as PBDB-T) or PBDB-T derivatives may be used. For example, a method of dispersing the electron donor material in the PBDB-T or the PBDB-T derivative may be used.

In one embodiment, the step of forming the network polymer may be performed by a room temperature process including a process using a shadow mask, including a printing method, a spin coating method, a screen printing method, and a doctor blade method.

In one embodiment, the crosslinking agent is a compound represented by Formula 4 below.

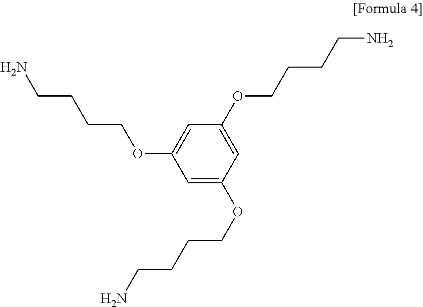 [SMILES: NCCCCOc1cc(OCCCCN)cc(OCCCCN)c1]

In one embodiment, the stoichiometric ratio of the monomer and the crosslinking agent is 1:0.33 to 1. More specifically, the stoichiometric ratio of the monomer and the crosslinking agent may be 1:0.33, 1:0.5, 1:0.66, 1:0.75, or 1:1, but is not limited thereto.

Hereinafter, the present invention will be described in detail with reference to preferred examples so that it can be easily carried out by one of ordinary skill in the art to which the present invention pertains. However, the present invention may be implemented in a variety of different forms and is not be limited to the examples described herein.

### EXAMPLES

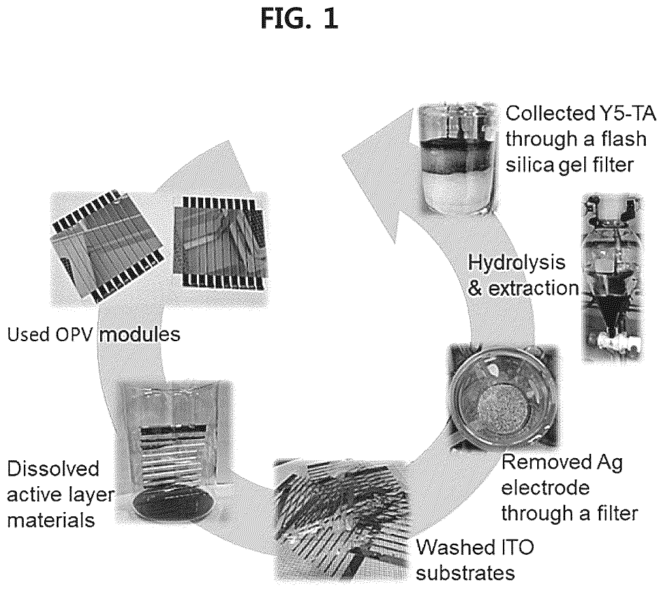

FIG. 1 is an image showing a process of recovering a monomer from a conjugated polymer according to an embodiment of the present invention.

### <Preparation Example 1> Recovery of Monomer from Recyclable Conjugated Polymer

After dissolving the photoactive layer of the organic photovoltaic (OPV) used using chloroform, the ITO substrate was washed and the silver (Ag) electrode was removed through a filter. Subsequently, PY-TIP (Poly[2-((1E,2Z)-2-((10-(((2Z,3E)-3-(cyano(isocyano)methylene)-5-(5-((E)-((4-(((E)-(5-methylthiophen-2-yl)methylene)amino) phenyl)imino) methyl)thiophen-2-yl)-1-oxo-1,3-dihydro-2H-inden-2-ylidene)methyl)-12,13-bis(2-octyldodecyl)-3,9-diundecyl-12,13-dihydro-[1,2,5]thiadiazolo[3,4-e]thieno[2″,3″: 4′,5′]thieno[2′,3′: 4,5]pyrrolo[3,2-g]thieno[2′,3′: 4,5]thieno[3,2-b]indol-2-yl)methylene)-6-methyl-3-oxo-2,3-dihydro-1H-inden-1-ylidene)-2-isocyanoacetonitrile]), which is the recyclable conjugated polymer, was hydrolyzed and extracted according to Scheme 1 below to recover Y5-TA (2,2′-((1E,1′E,2Z,2′Z)-((12,13-bis(2-octyldodecyl)-3,9-diundecyl-12,13-dihydro-[1,2,5]thiadiazolo[3,4-e]thieno[2″,3″: 4′,5′]thieno[2′,3′: 4,5]pyrrolo[3,2-g]thieno[2′,3′: 4,5]thieno[3,2-b]indole-2,10-diyl)bis(methaneylylidene)) bis(6-(5-formylthiophen-2-yl)-3-oxo-2,3-dihydro-1H-indene-2,1-diylidene))bis(2-isocyanoacetonitrile)), which is the monomer. The recovered monomer was filtered using flash silica gel.

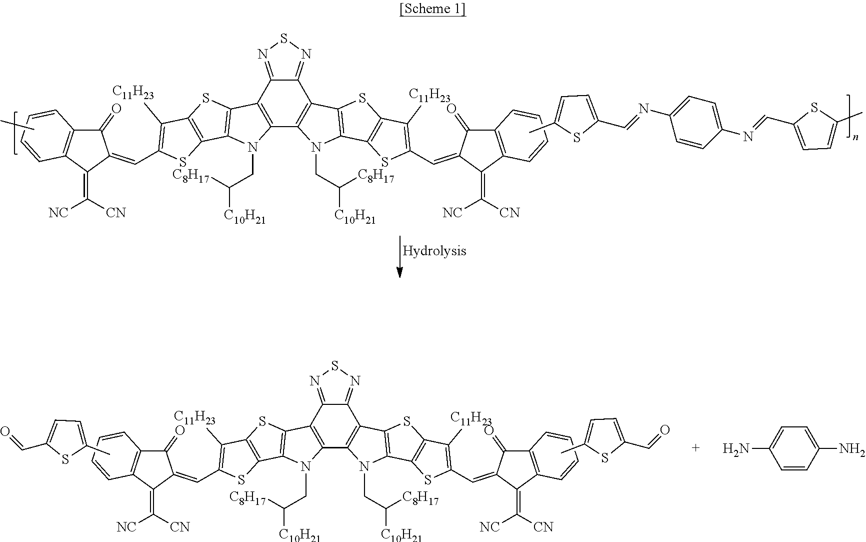

### <Preparation Example 2> Preparation of Network Polymer

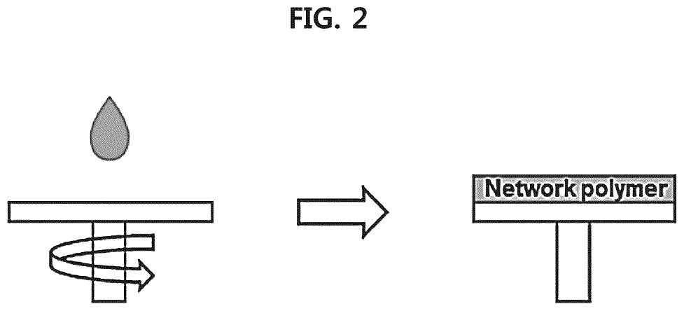

A mixture of Y5-TA, which is the monomer, and PO—N(4,4′,4″-(benzene-1,3,5-triyltris(oxy)) tris(butan-1-amine)), which is the cross-linking agents, was cast on a circular glass plate and dried to prepare PY-TIPO (Poly[2-((1E,2Z)-6-(5-((E)-((4-(3,5-bis(4-(methyleneamino) butoxy) phenoxy)butyl) imino)methyl) thiophen-2-yl)-2-((10-(((2Z,3E)-3-(cyano(isocyano)methylene)-1-oxo-5-(thiophen-2-yl)-1,3-dihydro-2H-inden-2-ylidene)methyl)-12,13-bis(2-octyldodecyl)-3,9-diundecyl-12,13-dihydro-[1,2,5]thiadiazolo[3,4-e]thieno[2″,3″: 4′,5′]thieno[2′,3′: 4,5]pyrrolo[3,2-g]thieno[2′,3′: 4,5]thieno[3,2-b]indol-2-yl)methylene)-3-oxo-2,3-dihydro-1H-inden-1-ylidene)-2-isocyanoacetonitrile]), which is the network polymer according to an embodiment of the present invention. FIG. 2 is an image showing a process of manufacturing a network polymer according to an embodiment of the present invention. The reaction of forming the network polymer was performed by Schiff base reaction according to [Scheme 2] below.

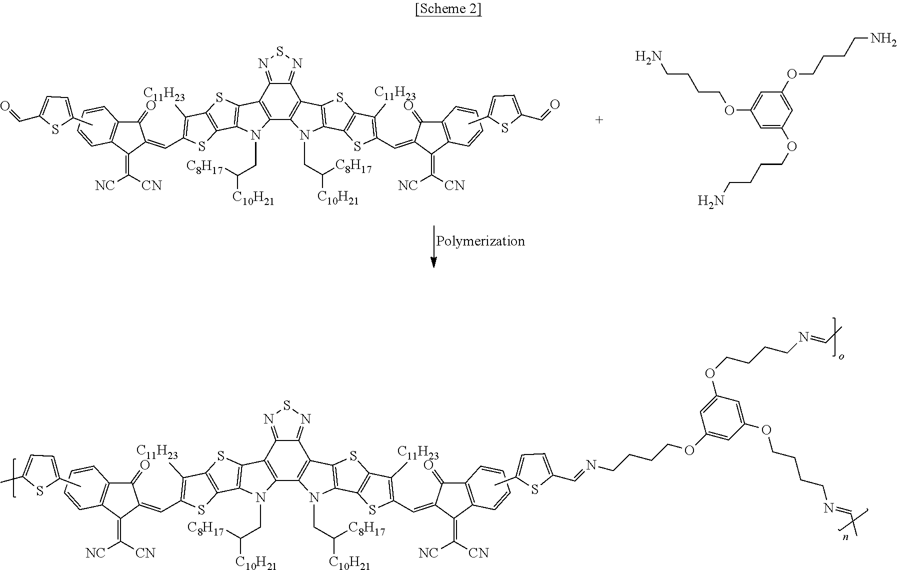

### <Preparation Example 3> Preparation of Organic Solar Cell

The structure of the manufactured bulk heterojunction type solar cell is as follows: ITO/Zinc oxide (ZnO)/Photoactive layer/Molybdenum oxide (MoO₃)/Ag. A zinc oxide solution was spin coated on a dried ITO substrate and then heat treated. Next, a photoactive layer composition containing the network polymer prepared in Preparation Example 2 was spin-coated on the zinc oxide-coated ITO substrate to form a photoactive layer. Molybdenum oxide was deposited on the photoactive layer to form an upper electrode, and a silver electrode was deposited on the molybdenum oxide.

### <Experimental Example 1> Evaluation of Formation Capacity of Network Polymer

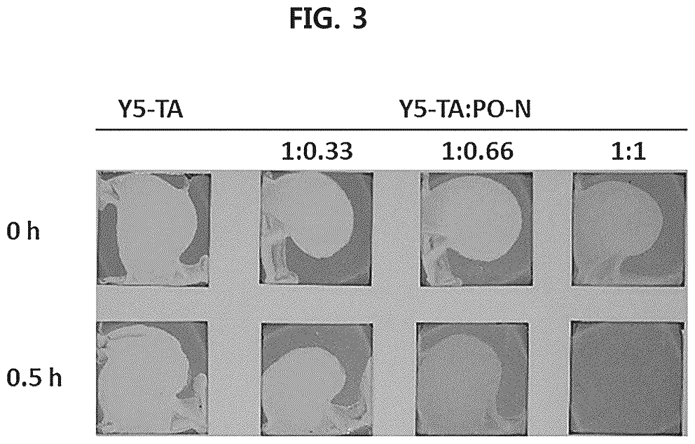

The network polymer prepared in Preparation Example 2 was washed using a solvent (dichloromethane), and then the formation capacity of the network polymer was evaluated. The results are shown in FIG. 3. FIG. 3 is an image showing the results of evaluating the formation capacity of a network polymer according to an embodiment of the present invention.

From FIG. 3, it can be seen that the network polymer according to an embodiment of the present invention is not washed away by the solvent and thus exhibits excellent formation capacity.

### <Experimental Example 2> Evaluation of Monomer Recovery Capacity 1

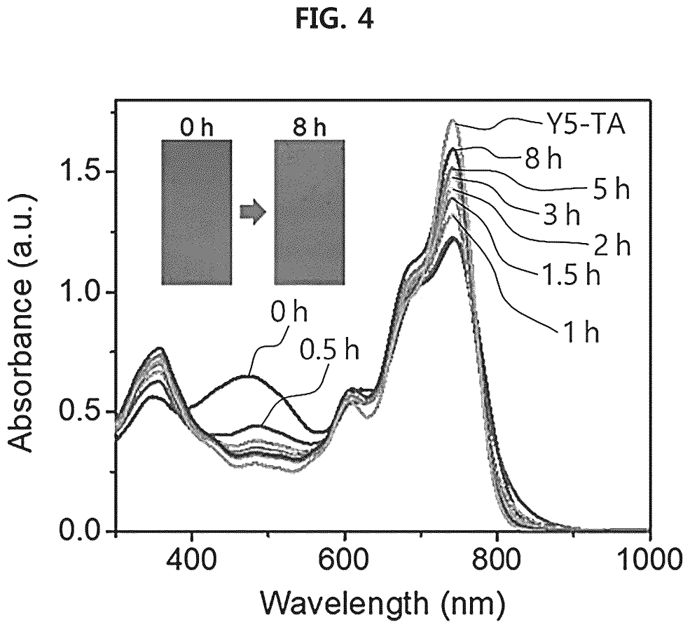

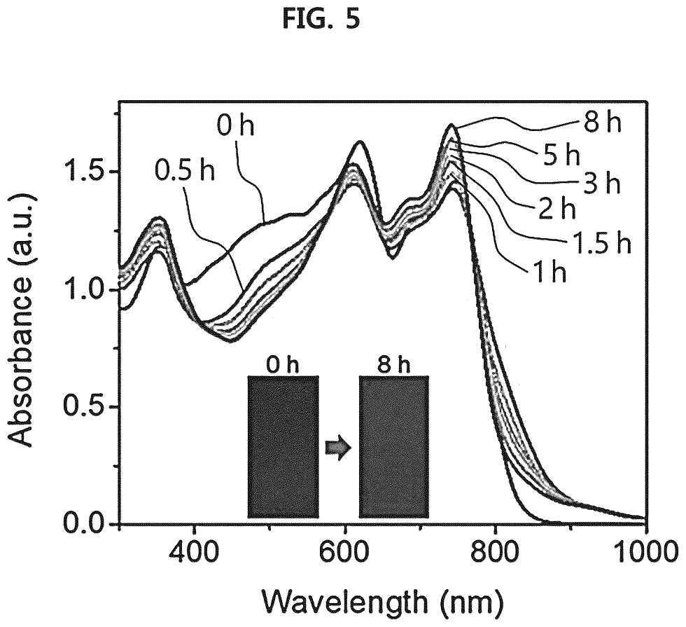

In the process of hydrolyzing the recyclable conjugated polymer in Preparation Example 1, the recovery capacity of the monomer was evaluated through UV-vis spectrum over time. The results are shown in FIGS. 4 and 5. FIGS. 4 and 5 are UV-vis spectrum graphs showing the results of evaluating the recovery capacity of a monomer according to an embodiment of the present invention.

From FIGS. 4 and 5, it can be seen by the unique peak that as time passes, the unique peak at 470 nm of the conjugated polymer gradually decreases, and the onset of the monomer near 800 nm shifts to blue. From this, it can be seen that the conjugated polymer is completely hydrolyzed into monomers within 8 hours.

### <Experimental Example 3> Evaluation of Monomer Recovery Capacity 2

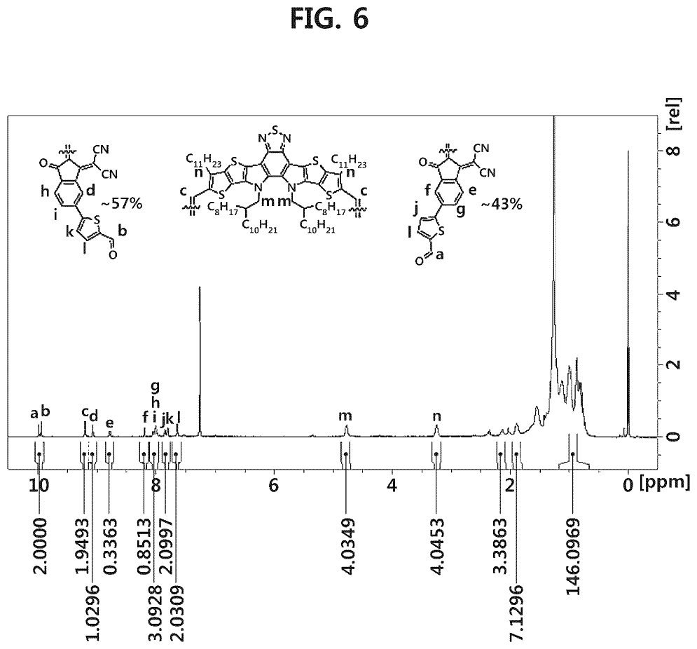

The recovery capacity of the monomer recovered in Preparation Example 1 was evaluated through NMR. The results are shown in FIG. 6. FIG. 6 is an NMR graph showing the results of evaluating the recovery capacity of a monomer according to an embodiment of the present invention.

From FIG. 6, it can be confirmed that the monomer recovered in Preparation Example 1 can be recovered without impurities. From this, it can be seen that the recovered monomer does not contain impurities and thus can be recycled without additional purification.

### <Experimental Example 4> Evaluation of Monomer Recovery Capacity 3

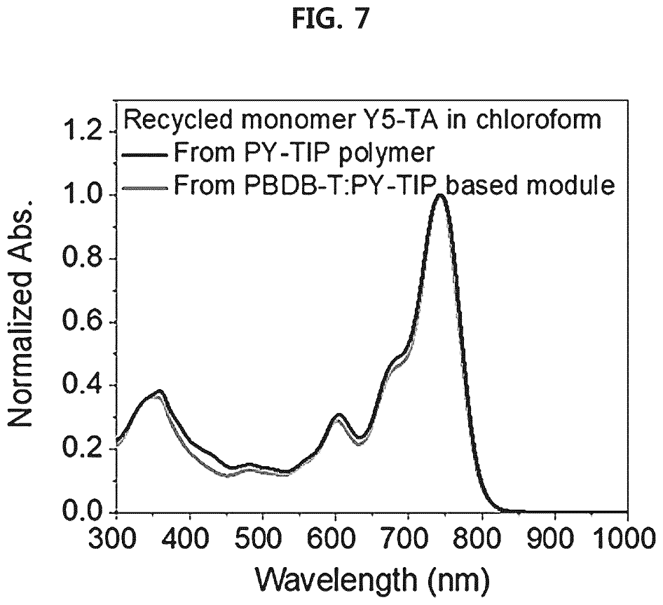

The recovery capacity of the monomer recovered in Preparation Example 1 was evaluated through UV-vis spectrum. The results are shown in FIG. 7. FIG. 7 is a UV-vis spectrum graph showing the results of evaluating the recovery capacity of a monomer according to an embodiment of the present invention.

From FIG. 7, it can be confirmed that the monomer recovered in Preparation Example 1 can be recovered without changing the optical properties of the material when compared to the monomer recovered from the existing conjugated polymer.

### <Experimental Example 5> Evaluation of Organic Solar Cell Efficiency

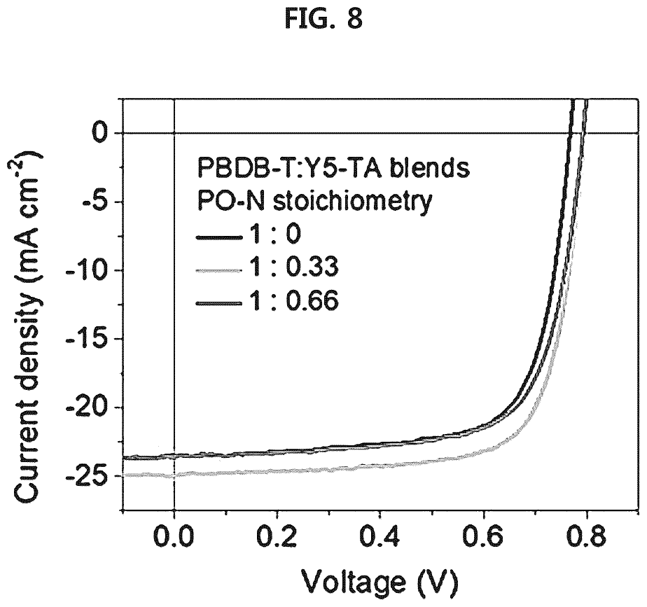

The energy conversion efficiency of the organic solar cell prepared in Preparation Example 3 was evaluated by measuring current density according to voltage. The results are shown in FIG. 8. FIG. 8 is a graph showing a current density-voltage (J-V) curve of an organic solar cell according to an embodiment of the present invention.

From FIG. 8, it can be confirmed that the organic solar cell prepared in Preparation Example 3 shows no decrease in performance at all, thereby exhibiting excellent efficiency.

### <Experimental Example 6> Evaluation of Thermal Stability of Organic Solar Cell

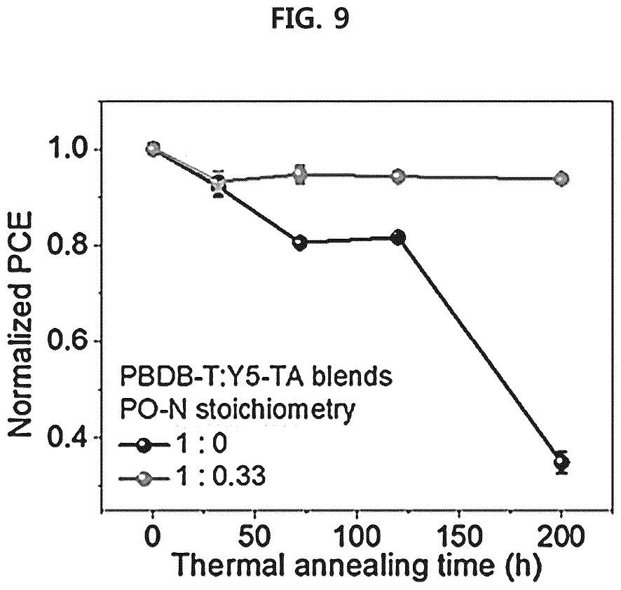

The thermal stability of the organic solar cell prepared in Preparation Example 3 was evaluated by measuring a change in power conversion efficiency (PCE) according to heating time (PCE(t)/PCE(0)) under a nitrogen atmosphere at 50° C. The results are shown in FIG. 9. FIG. 9 is a graph showing the results of evaluating the thermal stability of an organic solar cell according to an embodiment of the present invention.

From FIG. 9, it can be confirmed that the organic solar cell prepared in Preparation Example 3 has excellent thermal stability.

### <Experimental Example 7> Evaluation of Mechanical Properties of Network Polymer Thin Film

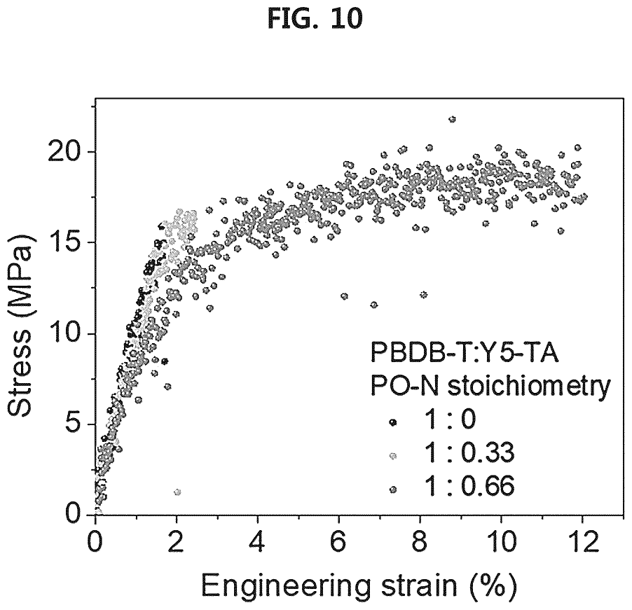

The mechanical properties of the thin film comprising the network polymer prepared in Preparation Example 2 were evaluated. The results are shown in FIG. 10. FIG. 10 is a graph showing the results of evaluating the mechanical properties of a network polymer according to an embodiment of the present invention. The x-axis in FIG. 10 is an engineering strain, which is an indicator of the amount by which the material is stretched in a tensile test. The y-axis is a stress, which means a load applied to the material in a tensile test divided by the cross-sectional area.

From FIG. 10, it can be confirmed that the thin film comprising the network polymer prepared in Preparation Example 2 has excellent tensile capability. In addition, it can be seen that the highest tensile capacity is shown when the stoichiometric ratio of the monomer and the crosslinking agent is 1:0.66. From this, it can be seen that the network polymer according to an embodiment of the present invention can be applied to flexible devices.

Although exemplary embodiments of the present invention have been described above in connection with the above-mentioned preferred examples, various modifications and variations can be made without departing from the gist and scope of the invention. Accordingly, the appended claims will include such modifications and variations as long as they fall within the gist of the present invention.

What is claimed is:

1. A network polymer formed from a monomer recovered from a recyclable conjugated polymer.

2. The network polymer according to claim 1, wherein the recyclable conjugated polymer includes an imine bond, and the monomer is a dialdehyde compound.

3. The network polymer according to claim 1, wherein the recyclable conjugated polymer includes a repeating unit represented by Formula 1 below: 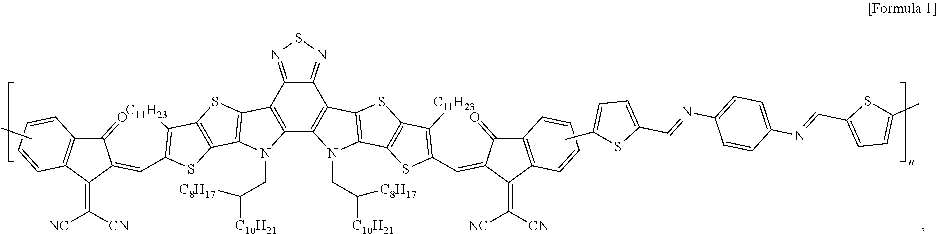
wherein n is an integer ranging from 1 to 1000.

4. The network polymer according to claim 1, wherein the monomer is represented by Formula 2 below:

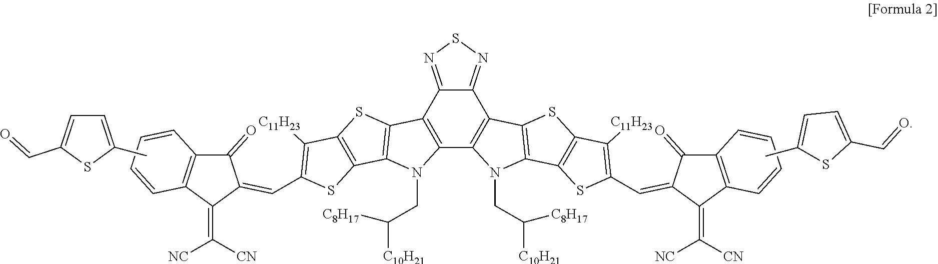

5. The network polymer according to claim 1, wherein the network polymer includes a repeating unit represented by Formula 3 below: 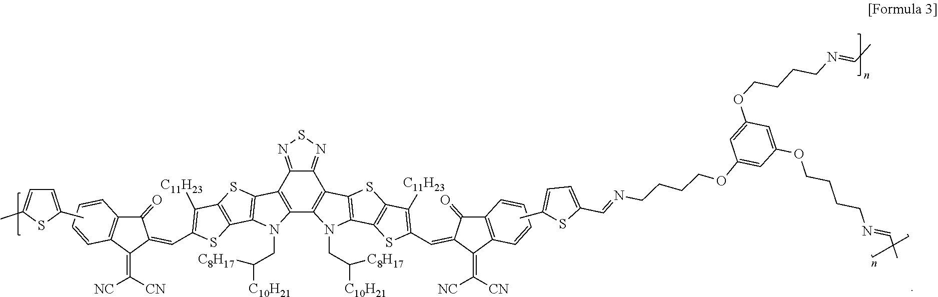
wherein n is an integer ranging from 1 to 1000.

6. An organic electronic device comprising the network polymer of claim 1.

7. The organic electronic device according to claim 6, wherein the organic electronic device is any one selected from an organic solar cell, an organic thin film transistor, and an organic light emitting diode.

8. The organic electronic device according to claim 6, wherein the organic electronic device is a bulk heterojunction (BHJ) type solar cell, and the network polymer is included in a photoactive layer of the bulk heterojunction type solar cell.
